(19) United States
(12) Patent Application Publication
(10) Pub. No.: US 2014/0275068 A1
(43) Pub. Date: Sep. 18, 2014

(54) PRO-DRUG COMPOUNDS

(71) Applicant: Proximagen Limited, London (GB)

(72) Inventors: Edward Savory, London (GB); Daniel Hill, Maple Grove, MN (US); Oldrich Kocian, Wadebridge (GB)

(73) Assignee: Proximagen Limited, London (GB)

(21) Appl. No.: 14/207,954

(22) Filed: Mar. 13, 2014

(30) Foreign Application Priority Data
Mar. 15, 2013 (GB) 1304814.5

(51) Int. Cl. C07D 311/68; C07D 405/12

(52) U.S. Cl. CPC C07D 311/68; C07D 405/12
USPC 514/233.5; 549/399; 514/456; 544/151; 548/525; 514/422; 546/187; 514/316

(57) ABSTRACT

The present invention provides neuronal gap junction blocking compounds according to formula (I) or a hydrate, solvate, or pharmaceutically acceptable salt thereof:

[chemical structure]

wherein the integers Q, R², A, R¹, Z₁, Z₂, and Z₃ are as defined in claim 1. The compounds are useful for the treatment or prevention of a range of conditions including, e.g., migraine, epilepsy, non-epileptic seizures, brain injury, and cardiovascular disease.

The present invention relates to neuronal gap junction blocking compounds having improved pharmacokinetic properties, the compounds being useful for the treatment or prevention of a range of conditions including migraine, epilepsy, non-epileptic seizures, brain injury (including stroke, intracranial haemorrhage and trauma induced) or cardiovascular disease including myocardial infarction, coronary revascularization or angina.

### BACKGROUND TO THE INVENTION

Cortical spreading depolarization (CSD) is a wave of depolarisation with consequent depressed electrical activity which spreads across the surface of the cerebral cortex (at a rate of 2-6 mm/min) usually followed by hyperaemia and neuronal hyperpolarisation. The reduction in electrical activity is a consequence of neuron depolarisation and swelling, with K+ efflux, Na and Ca influx and electrical silence. This abnormal neuronal activity is associated with delayed neuronal damage in a number of pathological states including cerebral ischaemia (arising from e.g. stroke, haemorrhage and traumatic brain injury Strong et al., 2002 Fabricius et al., 2006; Dreier et al., 2006 Dohmen et al., 2008), epilepsy and the aura associated with migraine (Lauritzen 1994; Goadsby 2007). As the CSD wave moves across the cortex it is associated with a reactive increase in local blood flow which may serve to help restore the more normal ionic balance of the neurons affected. After the CSD induced hyperaemia the local increase in blood flow attenuates (oligaemia) potentially resulting in imbalances in energy supply and demand. Under certain conditions, the reactive hyperaemia is not observed, but instead the local vasculature constricts resulting in ischaemia which in turn can lead to neuronal death. The conditions triggering this abnormal response in experimental models are high extracellular levels of K+ and low NO availability. These conditions are typically seen in ischaemic areas of the brain, and dusters of CSD waves in these circumstances result in spreading ischaemia (see Dreier 2011). Of particular importance is the spreading ischaemia seen after sub-arachnoid haemorrhage (SAH), in the penumbra of an infarct and after traumatic brain Injury where delayed neuronal damage can have a significant effect on clinical outcomes (Dreier et al., 2006, 2012; Hartings et al., 2011a, 2011b; Fabricius at al., 2006).

Given the detrimental effect of dusters of CSDs in humans and experimental animals, and the poor prognosis associated with CSDs, there is an unmet medical need for new compounds useful for inhibiting CSDs for patients with and without brain injuries. Without wishing to be bound by theory, the spread of CSD is believed to be mediated by gap junctions rather than by neuronal synaptic communication (Nedergard et al., 1995; Rawanduzy et al., 1997, Saito et al., 1997), the gap junctions providing a means of spreading the depolarisation in the absence of normal synaptic communication. Gap junctions are comprised of connexin proteins of which there are 21 in the human genome. Each Gap junction is made of two hemichannels, each comprising six connexin monomers.

Gap junctions are also implicated in a number of other disease states including hereditary diseases of the skin and ear (e.g. keratitis-ichthyosis deafness syndrome, erythrokeratoderma variabilis, Vohwinkel's syndrome, and hypotrichosis-deafness syndrome). Blockade of gap junction proteins has been shown to beneficial in some preclinical models of pain (e.g. Spataro et al., 2004 J Pain 5, 392-405, Wu at al., 2012 J Neurosci Res. 90, 337-45). This is believed to be a consequence of gap junction blockade in the spinal cord resulting in a reduction in the hypersensitivity of the dorsal horn to sensory nerve input. In addition gap junctions and their associated hemichannels have been implicated in neurodegenerative diseases including Alzheimer's disease, Parkinson's Disease, Huntington's Disease and amyotrophic lateral sclerosis (Takeuchi et al 2011 PLoS One.; 6, e21108).

Tonabersat (SB-220453/PRX201145) is a gap junction blocker (Silberstein, 2009; Durham and Garrett, 2009) which binds selectively and with high affinity to a unique stereo-selective site in rat and human brains. Consistent with its action on gap junctions Tonabersat also inhibits high K+ evoked CSD in cats (Smith et al., 2000; Read et al., 2000; Bradley et al., 2001) and rats (Read et al., 2001).

However, known gap junction blockers, including Tonabersat and Carabersat, suffer from undesirable physiochemical properties. Tonabersat is a crystalline solid with a high melting point (152-153C) and with a relatively high lipophilicity (log P 3.32). The compound has no readily ionisable groups and consequently has a low aqueous solubility of 0.025 mg/ml over a range of pH values including pH of 7.4. The low aqueous solubility of Tonabersat makes both intravenous (IV) and oral (PO) modes of administration problematic. The poor aqueous solubility prevents rapid injection of the required dose of Tonabersat which is required for the treatment of head injuries and stroke or for emergency treatment of epileptic seizures where the patient may be unconscious and unable to swallow an oral drug. At present the effective plasma concentrations needed to reduce the cortical spreading depression caused by head injury or stroke can only be reached by slow IV infusion given over a period of hours. With respect to the PO administration of Tonabersat for the treatment of other indications, solubility limited dissolution of the tablet form of Tonabersat given PO leads to a significant “food effect” with differences in the maximum blood concentration of Tonabersat (Cmax) seen depending on whether the drug is given with or without food. These differences make it difficult to accurately predict the plasma exposure of Tonabersat when given orally, thus increasing the risk of under or over dosing the patient.

Therefore it is an object of the present invention to provide gap junction blocker compounds having improved physiochemical properties thus improving the utility of these agents in treating a range of disease states.

### BRIEF DESCRIPTION OF THE INVENTION

The present invention makes available three classes of compounds, each class having one or more solubilising pro-drug groups.

### DETAILED DESCRIPTION OF THE INVENTION

In a first aspect, the present invention makes available a class of compounds of formula (I) or a hydrate, solvate, or pharmaceutically acceptable salt thereof:

[chemical structure]

wherein

Z₁, Z₂, and Z₃ are each independently selected from H, F, or Cl,

Q is O,

R² is H,

A is a direct bond, —C(O)O*—, C(R³XR⁴)O*—, —C(O)NH* wherein the atom marked * is directly connected to R¹,

R³ and R⁴ are selected independently from H, fluoro, C₁₋₄ alkyl, or C₁₋₄ fluoroalkyl, or R³ and R⁴ together with the atom to which they are attached form a cyclopropyl group, R¹ is selected from groups [1], [2], [3], [4], [5], [6], [7], [8], [9] or [10] wherein the atom marked ** is directly connected to A:

[chemical structure]

n is 0, 1, 2, or 3,

R⁵ is hydrogen,

R⁶ is selected from —CH₂CH(OH)CH₂OH, or —CH₂CH₂R⁹;

R⁷ and R⁷⁰ are independently selected from H, C₁₋₄ alkyl, or C₁₋₄ fluoroakyl;

R⁸ and R^{8b} are selected from:

(i) H, C₁₋₄ alkyl, or C₁₋₄ fluoroalkyl, or

(ii) the side chain of a natural or unnatural alpha-amino acid;

or R⁷ and R⁸ together with the atom to which they are attached form a C₃₋₇ carbocyclic ring;

R⁹ is selected from —N(R¹¹XR¹²), or —N⁺(R¹¹)(R¹²)(R¹³)X⁻, N(R¹¹)C(O)R¹⁴, —SO₃H or —OP(O)(OH)₂;

wherein R¹¹, R¹², and R¹³ are independently selected from H, C₁₋₄ alkyl, or C₁₋₄ fluoroalkyl, or R¹¹ and R¹² together with the nitrogen atom to which they are attached form a 4-7 membered heterocyclic ring optionally substituted with one or more groups selected from H, fluoro, C₁₋₄ alkyl, C₁₋₄ fluoroalkyl, C₁₋₄ alkoxy, or —C(O)R₃;

or in the case where R¹ is group [7], R⁹ is —NR¹¹R¹², wherein R¹¹ is hydrogen C₁₋₄ alkyl, or C₁₋₄ fluoroalkyl, and R¹² is C₁₋₄ alkyl, or C₁₋₄ fluoroalkyl, and wherein R¹² joins together with R^{8b} such that R¹² and R^{8b} together with the nitrogen to which R¹² is attached form a 5 or 6 membered cyclic amine group;

R¹⁴ is H, C₁₋₄ alkyl, or C₁₋₄ fluoroalkyl;

X⁻ is a pharmaceutically acceptable anion;

R¹⁵ is 3-pyridyl or 1,4-dihydro-1-methyl-pyridin-3-yl;

Y is —O—, —CH₂, —N(H)—, or —N(CH₃)—.

In a second aspect, the present invention makes available a class of compounds of formula (II) or a hydrate, solvate, or pharmaceutically acceptable salt thereof:

[chemical structure]

wherein

Z₁, Z₂, and Z₃ are each independently selected from H, F, or Cl,

Q is O,

A is a direct bond and R¹ is H,

R² is B-R²¹ wherein,

B is a direct bond, —C(O)O*—, C(R²³)(R²⁴)O*—, —C(O)NH* wherein the atom marked * is directly connected to R²¹,

R²³ and R²⁴ are selected independently from hydrogen, fluoro, C₁₋₄ alkyl, or C₁₋₄ fluoroalkyl, or R²³ and R²⁴ together with the atom to which they are attached form a cyclopropyl group,

R²¹ is selected from groups [1], [2], [3], [4], [5], [6], [7], [8], [9] or [10] wherein the atom marked ** is directly connected to B:

[chemical structure]

n is 0, 1, 2, or 3,

R⁵ is hydrogen,

R⁶ is selected from —CH₂CH(OH)CH₂OH, or —CH₂CH₂R⁹;

R⁷ and R^{7b} are independently selected from H, C₁₋₄ alkyl, or C₁₋₄ fluoroalkyl;

R⁸ and R^{8b} are selected from:

(i) H, C₁₋₄ alkyl, or C₁₋₄ fluoroalkyl, or

(ii) the side chain of a natural or unnatural alpha-amino acid;

or R⁷ and R⁸ together with the atom to which they are attached form a C₃₋₇ carbocyclic ring;

R⁹ is selected from —N(R¹¹)(R¹²), or —N⁺(R¹¹)(R¹²)(R¹³)X⁻, N(R¹¹)C(O)R¹⁴, —SO₃H or —OP(O)(OH)₂;

wherein R¹¹, R¹², and R¹³ are independently selected from H, C₁₋₄ alkyl, or C₁₋₄ fluoroalkyl, or

R¹¹ and R¹² together with the nitrogen atom to which they are attached form a 4-7 membered heterocyclic ring optionally substituted with one or more groups selected from H, fluoro, C₁₋₄ alkyl, C₁₋₄ fluoroalkyl, C₁₋₄ alkoxy, or —C(O)R₃;

or in the case where R¹ is group [7], R⁹ is —NR¹¹R¹², wherein R¹¹ is hydrogen, C₁₋₄ alkyl, or C₁₋₄ fluoroalkyl, and R¹² is C₁₋₄ alkyl, or C₁₋₄ fluoroalkyl, and wherein R¹² joins together with R^{8b} such that R¹² and R^{8b} together with the nitrogen to which R¹² is attached form a 5 or 6 membered cyclic amine group;

R¹⁴ is H, C₁₋₄ alkyl, or C₁₋₄ fluoroalkyl;

X⁻ is a pharmaceutically acceptable anion,

R¹⁵ is 3-pyridyl or 1,4-dihydro-1-methyl-pyridin-3-yl;

Y is —O—, —CH₂, —N(H)—, or —N(CH₃)—.

In a third aspect, the present invention makes available a class of compounds of formula (IIIa) or (IIIb), or a hydrate, solvate, or pharmaceutically acceptable salt thereof:

[chemical structure]

wherein Z₁, Z₂, and Z₃ are each independently selected from H, F, or Cl; and

R² and -A-R¹ are both H; and

In the case of formula (IIIa):

R⁴¹ and R⁴² are independently H, C₁₋₄ fluoroalkyl or optionally substituted C₁₋₄ alkyl, or R⁴¹ and R⁴² together with the carbon atom to which they are attached form a 5-8 membered heterocycle, any carbon atom of which is optionally substituted; or

In the case of formula (IIIb):

Q is an oxime of formula ═NHOR⁴³, wherein R⁴³ is

(i) selected from H, C₁₋₄ fluoroalkyl or optionally substituted C₁₋₄ alkyl, or

(ii) -A-R¹ wherein

A is a direct bond, —C(O)O*—, C(R³)(R⁴)O*—, —C(O)NH* wherein the atom marked * is directly connected to R¹;

R³ and R⁴ are selected independently from H, fluoro, C₁₋₄ alkyl, or C₁₋₄ fluoroalkyl, or R³ and R⁴ together with the atom to which they are attached form a cyclopropyl group,

R¹ is selected from groups [1], [2], [3], [4], [5], [6], [7], [8], [9] or [10] wherein the atom marked ** is directly connected to A:

[chemical structure]

n is 0, 1, 2, or 3;

R⁵ is hydrogen;

R⁶ is selected from —CH₂CH(OH)CH₂OH, or —CH₂CH₂R⁹;

R⁷ and R^{7b} are independently selected from H, C₁₋₄ alkyl, or C₁₋₄ fluoroalkyl;

R⁸ and R^{8b} are selected from:

(i) H, C₁₋₄ alkyl, or C₁₋₄ fluoroalkyl, or

(ii) the side chain of a natural or unnatural alpha-amino acid;

or R⁷ and R⁸ together with the atom to which they are attached form a C₃₋₇ carbocyclic ring;

R⁹ is selected from —N(R¹¹)(R¹²), or —N⁺(R¹¹)(R¹²)(R¹³)X⁻, N(R¹¹)C(O)R¹⁴, —SO₃H or —OP(O)(OH)_{z};

wherein R¹¹, R¹², and R¹³ are independently selected from H, C₁₋₄ alkyl, or C₁₋₄ fluoroalkyl, or

R¹¹ and R¹² together with the nitrogen atom to which they are attached form a 5-7 membered heterocyclic ring optionally substituted with one or more groups selected from H, fluoro, C₁₋₄ alkyl, C₁₋₄ fluoroalkyl, C₁₋₄ alkoxy, or —C(O)R₃;

or in the case where R¹ is group [7], R⁹ is —NR¹¹R¹², wherein R¹¹ is hydrogen C₁₋₄ alkyl, or C₁₋₄ fluoroalkyl, and R¹² is C₁₋₄ alkyl, or C₁₋₄ fluoroalkyl, and wherein R¹² joins together with R^{8b} such that R¹² and R^{8b} together with the nitrogen to which R1² is attached form a 5 or 6 membered cyclic amine group;

R¹⁴ is H, C₁₋₄ alkyl, or C₁₋₄ fluoroalkyl;

X⁻ is a pharmaceutically acceptable anion;

R¹⁵ is 3-pyridyl or 1,4-dihydro-1-methyl-pyridin-3-yl;

Y is —O—, —CH₂—, —N(H)—, or —N(CH₃)—.

In an embodiment R⁴³ is C₁₋₄ alkyl optionally substituted with a phosphate group (—P(O)OR⁶¹OR⁶²). In an example of such an embodiment OR⁴³ is —OCH₂P(O)OR⁶¹OR⁶², wherein R⁶¹ and R⁶² are independently H or C₁₋₄ alkyl.

In another embodiment R⁴³ is an amino acid derivative having the structure —C(O)CH(R¹⁰⁰)NH₂ wherein the group R¹⁰⁰ is the side chain of a natural or unnatural amino acid. In an embodiment OR⁴³ is —OC(O)CH(CH(CH₃)₂)NH₂.

Preferably the invention is as set out in the claims.

### TERMINOLOGY

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein, the term “(Cₐ-C_{b})fluoroalkyl” has the same meaning as “(Cₐ-C_{b})alkyl” except that one or more of the hydrogen atoms directly connected to the carbon atoms forming the alkyl group is replaced by the corresponding number of fluorine atoms.

As used herein the unqualified term “carbocyclic” refers to a mono-, bi- or tricyclic radical having up to 16 ring atoms, all of which are carbon, and includes aryl and cycloalkyl.

As used herein the unqualified term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthlenyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, triazinyl, indolyl and indazolyl.

As used herein the unqualified term “heterocycyl” or “heterocyclic” includes “heteroaryl” as defined above, and in addition means a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

When the term cyclic amino group is used the cyclic amino groups can have 3-8 ring atoms, 3-7 ring atoms, 5-7 ring atoms, 5-6 ring atoms. When the terms 3-8 or 3-7 cyclic amino group is used all ranges within those ranges are disclosed, for example 3-8 includes 3-7. Both 3-8 and 3-7 include 4-7 and 5-7 and 5-6. Examples of 5 and 6 membered cyclic amino groups include morpholine, piperidine, piperazine, pyrrolidine.

Unless otherwise specified in the context in which it occurs, the term “substituted” as applied to any moiety herein means substituted with up to four compatible substituents, each of which independently may be, for example, (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro, bromo and chloro), fully or partially fluorinated (C₁-C₃)alkyl, (C₁-C₃)alkoxy or (C₁-C₃)alkylthio such as trifluoromethyl, trifluoromethoxy, and trifluoromethylthio, nitro, nitrile (—CN), oxo, phenyl, phenoxy, monocyclic heteroaryl or heteroaryloxy with 5 or 6 ring atoms, tetrazolyl, —COOR^{A}, —COR^{A}, —OCOR^{A}, —SO₂R^{A}, —CONR^{A}R^{B}, —SO₂NR^{A}R^{B}, —NR^{A}R^{B}, OCONR^{A}R^{B}, —NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl group or, in the case where R^{A} and R^{B} are linked to the same N atom, R^{A} and R^{B} taken together with that nitrogen may form a cyclic amino ring, such as a morpholine, piperidinyl or piperazinyl ring. Where the substituent is phenyl, phenoxy or monocyclic heteroaryl or heteroaryloxy with 5 or 6 ring atoms, the phenyl or heteroaryl ring thereof may itself be substituted by any of the above substituents except phenyl, phenoxy, heteroaryl or heteroaryloxy. An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds of formula (I), (II), (IIIa) or (IIIb) which are basic can form salts, including pharmaceutically acceptable salts with inorganic acids, e.g. hydrohalic acids such as hydrochloric or hydrobromic acids, sulphuric acid, nitric acid or phosphoric acid and the like, and with organic acids e.g. acetic, tartaric, succinic, fumaric, maleic, malic salicylic, citric, methanesulphonic, p-toluenesulphonic, benzoic, benzenesunfonic, glutamic, lactic, and mandelic acids and the like.

The formation of specific salt forms can provide compounds of the invention with improved physicochemical properties. For a review on suitable salts, see Handbook of Pharmaceutical Salts: Properties, Selection, and Use by Stahl and Wermuth (Wiley-VCH, Weinheim, Germany, 2002).

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water. Compounds with which the invention is concerned which may exist in one or more stereoisomeric form, because of the presence of asymmetric atoms or rotational restrictions, can exist as a number of stereoisomers with R or S stereochemistry at each chiral centre or as atropisomers with R or S stereochemistry at each chiral axis. The invention includes all such enantiomers and diastereoisomers and mixtures thereof. In particular the carbon atom to which the R⁸ or R^{8b} substituent is attached may be in either the R or the S stereochemical configuration.

The compounds of the invention include compounds of formula (I), (II), (IIIa) or (IIIb) as hereinbefore defined, including all polymorphs and crystal habits thereof, prodrugs and isomers thereof (including optical, geometric and tautomeric isomers) as hereinafter defined and isotopically-labeled compounds of formula (I), (II), (IIIa) or (IIIb).

Also included within the scope of the invention are metabolites of compounds of formula (I), (II), (IIIa) or (IIIb), that is, compounds formed in vivo upon administration of the drug. Some examples of metabolites include:
(i) where the compound of formula I contains a methyl group, an hydroxymethyl derivative thereof (—CH₃->—CH₂OH):
(ii) where the compound of formula I contains an alkoxy group, an hydroxy derivative thereof (—OR->—OH);
(iii) where the compound of formula I contains a tertiary amino group, a secondary amino derivative thereof (e.g. —NR^{1A}R^{2A}->—NHR^{1A} or —NHR^{2A});
(iv) where the compound of formula I contains a secondary amino group, a primary derivative thereof (—NHR^{1A}->—NH₂);
(v) where the compound of formula I contains a phenyl moiety, a phenol derivative thereof (-Ph->-PhOH); and
(vi) where the compound of formula I contains an amide group, a carboxylic acid derivative thereof (—CONH₂->COOH).

For use in accordance with the invention, the following structural characteristics are currently contemplated, in any compatible combination, in the compounds of formula (I):

The groups Z₁, Z₂, and Z₃ are each independently selected from H, F, or Cl. In an embodiment Z₁ is Cl, Z₂ is F, and Z is H. In another embodiment Z₁ is Cl, Z and Z₃ are H. In another embodiment Z₁ is H, Z₂ is F, and Z₃ is H. In another embodiment Z₁ is F, Z₂ is H, and Z₃ is F.

The above definitions of Z₁, Z₂, and Z₃ is H are applicable to compounds of formula (I), (II), (IIIa), and (IIIb). As an illustration, the preferred definition of Z₁, Z₂, and Z₃ applied to the compounds of formula (I) is as follows:

[chemical structure]

In a preferred embodiment Z₁ is Cl. Z₂ is F or H, and Z₃ is H.

Examples of side chains of natural alpha amino acids include those of alanine, arginine, asparagine, aspartic acid, cysteine, cystine, glutamic acid, histidine, 5-hydroxylysine, 4-hydroxyproline, isoleucine, leucine, lysine, methionine, phenylalanine, proline, serine, threonine, tryptophan, tyrosine, valine, α-aminoadipic acid, α-amino-n-butyric acid, 3,4-dihydroxyphenylalanine, homoserine, α-methylserine, ornithine, pipecolic acid, and thyroxine.

Natural alpha-amino acids which contain functional substituents, for example amino, carboxyl, hydroxy, mercapto, guanidyl, imidazolyl, or indolyl groups in their characteristic side chains include arginine, lysine, glutamic acid, aspartic acid, tryptophan, histidine, serine, threonine, tyrosine, and cysteine. When R⁸ or R^{8b} in the compounds of the invention is one of those side chains, the functional substituent may optionally be protected.

The term “protected” when used in relation to a functional substituent in a side chain of a natural alpha-amino acid means a derivative of such a substituent which is substantially non-functional. For example, carboxyl groups may be esterified (for example as a C₁-C₆ alkyl ester), amino groups may be converted to amides (for example as a NHCOC₁-C₆ alkyl amide) or carbamates (for example as an NHC(═O)OC₁-C₆ alkyl or NHC(═O)OCH₂Ph carbamate), hydroxyl groups may be converted to ethers (for example an OC₁-C₆ alkyl or a O(C₁-C₆ alkyl)phenyl ether) or esters (for example a OC(═O)C₁-C₆ alkyl ester) and thiol groups may be converted to thioethers (for example a tert-butyl or benzyl thioether) or thioesters (for example a SC(═O)C₁-C₆ alkyl thioester).

Examples of side chains of non-natural alpha amino acids include:

an optional substituent, C₁-C₆ alkyl, phenyl, 2, -3-, or 4-hydroxyphenyl, 2, -3-, or 4-methoxyphenyl, 2, -3-, or 4-pyridylmethyl, benzyl, phenylethyl, 2-, 3-, or 4-hydroxybenzyl, 2, -3-, or 4-benzyoxybenzyl, 2, -3-, or 4-C₁-C₆ alkoxybenzyl, and benzyloxy(C₁-C₆ alkyl)-groups, wherein any of the foregoing non-natural amino acid side chains is optionally substituted in the alkyl, phenyl or pyridyl group; or

groups -[Alk]ₙR₅₀ where Alk is a (C₁-C₆)alkyl or (C₂-C₆)alkenyl group optionally interrupted by one or more —O—, or —S— atoms or —N(R₅₁)— groups [where R₅₁ is a hydrogen atom or a (C₁-C₆)alkyl group], n is 0 or 1, and R₅₀ is an optionally substituted cycloalkyl or cycloalkenyl group; or

a heterocyclic(C₁-C₆)alkyl group, either being unsubstituted or mono- or di-substituted in the heterocyclic ring with halo, nitro, carboxy, (C₁-C₆)alkoxy, cyano, (C₁-C₆)alkanoyl, trifluoromethyl (C₁-C₆)alkyl, hydroxy, formyl, amino, (C₁-C₆)alkylamino, di-(C₁-C₆)alkylamino, mercapto, (C₁-C₆)alkylthio, hydroxy(C₁-C₆)alkyl, mercapto(C₁-C₆)alkyl or (C₁-C₆)alkylphenylmethyl; and

The group A

A is a direct bond, —C(O)O*—, C(R³)(R⁴)O*— such as —CH₂O—, CH(CH₃)O—, or C(CH₃)₂O—, —C(O)NH* wherein the atom marked * is directly connected to R¹,

R³ and R⁴ are selected independently from H, fluoro, C₁₋₄ alkyl such as methyl, ethyl or isopropyl, or C₁₋₄ fluoroalkyl such as trifluoromethyl, or R³ and R⁴ together with the atom to which they are attached form a cyclopropyl group. In an embodiment R³ and R⁴ are both hydrogen.

The group R¹

R¹ is selected from any one of the groups [1], [2], [3], [4], [5], [6], [7], [8], [9] or [10] wherein the atom marked ** is directly connected to A:

[chemical structure]

n is 0, 1, 2, 3

R⁵ is hydrogen

R⁶ is selected from —CH₂CH(OH)CH₂OH, or —CH₂CH₂R⁹. In an embodiment R⁶ is —CH₂CH(OH)CH₂OH, —CH₂CH₂NR¹¹R¹², or —CH₂CH₂NR¹¹R¹²R¹³X⁻. In an embodiment R¹¹ and R¹² together with the nitrogen atom to which they are attached form a 5, 6, or 7 membered cyclic amino group such as pyrrolidine, piperidine, homopiperazine, piperazine, homopiperazine, morpholine, or homomorpholine.

R⁷ and R^{7b} are independently selected from hydrogen, C₁₋₄ alkyl such as methyl, ethyl, isopropyl, or C₁₋₄ fluoroalkyl such as trifluoromethyl. In an embodiment R⁷ and R^{7b} are both hydrogen.

R⁸ and R^{8b} are selected from:
(iii) H, C₁₋₄ alkyl, or C₁₋₄ fluoroalkyl, or
(iv) the side chain of a natural or unnatural alpha-amino acid;

or R⁷ and R⁸ together with the atom to which they are attached form a C₃₋₇ carbocyclic ring;

R⁹ is selected from —N(R¹¹)(R¹²) such as —N(CH₃)₂, or —N⁺(R¹¹)(R¹²)(R¹³)X⁻, —N(R¹¹)C(O)R¹⁴, —SO₃H or —OP(O)(OH)₂;

wherein R¹¹, R¹², and R¹³ are independently selected from H, C₁₋₄ alkyl, or C₁₋₄ fluoroalkyl. In an embodiment R¹¹, R¹², and R¹³ are methyl or ethyl.

In an embodiment R¹¹ and R¹² together with the nitrogen atom to which they are attached form a 4 to 7 membered heterocyclic ring optionally substituted with one or more groups selected from H, fluoro, C₁₋₄ alkyl such as methyl or isopropyl, C₁₋₄ fluoroalkyl, C₁₋₄ alkoxy such as methoxy, or —C(O)R₃ such as —C(O)CH₃.

In an embodiment R¹ is group [7], R⁹ is —NR¹¹R¹², wherein R¹¹ is hydrogen, C₁₋₄ alkyl, or C₁₋₄ fluoroalkyl, and R¹² is C₁₋₄ alkyl, or C₁₋₄ fluoroakyl, and the group R¹² joins together with R^{8b} or the carbon atom to which R^{8b} is attached such that R¹² and R^{8b} together with the nitrogen atom to which R¹² is attached form a 5 or 6 membered cyclic amine group. In an embodiment that ring formed by R^{8b} and R¹² is a 5-membered ring such that the amino acid proline is formed. The ring of the proline amino acid is optionally substituted with one or more groups selected from H, fluoro, C₁₋₄ alkyl such as methyl or isopropyl, C₁₋₄ fluoroalkyl, C₁₋₄ alkoxy such as methoxy, or —C(O)R₃ such as —C(O)CH.

R¹⁴ is H, C₁₋₄ alkyl such as methyl, or C₁₋₄ fluoroalkyl;

X⁻ is a pharmaceutically acceptable anion;

R¹⁵ is 3-pyridyl or 1,4-dihydro-1-methyl-pyridin-3-yl;

Y is —O—, —CH₂—, —N(H)—, or —N(CH₃)—.

In an embodiment R¹ is selected from any one of groups [4A], [4B], [4C], [4D], [5A], or [5B]:

[chemical structure]

Specific compounds of the invention include those of the Examples herein.

It will be understood that the compounds of formula (I), (II), (IIIa) or (IIIb) may be further modified by adding one or more of the prodrug groups Q, -AR¹ or R². For example the compounds of formula (I) or (II) may be modified by exchanging the oxygen atom Q for a prodrug Q group as defined in (IIIa) or (IIIb). Alternatively, the compounds of formula (I) could be modified by replacing the hydrogen atom R² by the prodrug group R² as defined in formula (II), and vice versa.

The present invention makes available a pharmaceutical composition comprising a compound of formula (I), (II), (IIIa) or (IIIb) together with one or more pharmaceutically acceptable carriers and/or excipients.

The present invention makes available a compound of formula (I), (II), (IIIa) or (IIIb) for use in medicine.

In an embodiment the inventions encompasses the use of a compound of formula (I), (II), (IIIa) or (IIIb) treatment of a disease or medical condition which benefits from inhibition of gap junction activity. Inhibition of gap junction activity may be achieved by blocking the gap junction as a whole or by blocking one or more hemichannels.

In an embodiment the inventions encompasses a method of treatment of a disease or medical condition which benefits from Inhibition of gap junction activity, comprising administering to a subject suffering from such disease or condition and effective amount of a compound of formula (I), (II), (IIIa) or (IIIb).

In an embodiment the disease or condition which benefits from inhibition of gap junction activity is selected from among migraine, aura with or without migraine, epilepsy, non-epileptic seizures, cerebrovascular accidents including stroke, intracranial haemorrhage (including traumatic brain injury, epidural hematoma, subdural hematoma and subarachnoid haemorrhage), and intra-cerebral haemorrhage, spinal cord vascular accidents arising from trauma, epidural hematoma, subdural hematoma or subarachnoid haemorrhage, pain including pain arising from hyperalgesia caused by damage to sensory neurons (i.e. neuropathic pain including but not limited to diabetic neuropathy, polyneuropathy, cancer pain, fibromyalgia, myofascial pain, post herpetic neuralgia, spinal stenosis, HIV pain, post-operative pain, post-trauma pain) or inflammation (including pain associated with osteoarthritis, rheumatoid arthritis, sciatica/radiculopathy, pancreatitis, tendonitis), neurodegenerative disease (including but not limited to Alzheimer's Disease, Parkinson's Disease, Huntington's Disease and Amyotrophic Lateral Sclerosis) and cardiovascular disease including myocardial infarction, coronary revascularization or angina.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art. However, for administration to human patients, the total daily dose of the compounds of the invention may typically be in the range 1 mg to 1000 mg depending, of course, on the mode of administration. For example, oral administration may require a total daily dose of from 10 mg to 1000 mg, while an intravenous dose may only require from 1 mg to 500 mg. The total daily dose may be administered in single or divided doses and may, at the physician's discretion, fall outside of the typical range given herein.

These dosages are based on an average human subject having a weight of about 60 kg to 100 kg. The physician will readily be able to determine doses for subjects whose weight falls outside this range, such as infants and the elderly, and especially obese patients.

The compounds with which the invention is concerned may be prepared for administration by any route consistent with their pharmacokinetic properties. Suitable routes for administration include oral, intravenous, buccal, intranasal, inhalation, rectal, and intradermal. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

The pro-drug may also be administered parenterally in a sterile medium. Depending on the vehicle and concentration used, the drug can either be suspended or dissolved in the vehicle.

Advantageously, adjuvants such as local anaesthetic, preservative and buffering agents can be dissolved in the vehicle. The person skilled in the art is aware of many excipients useful for IV formulation.

### Preparation of Compounds of the Invention

The compounds of formula (I) above may be prepared by, or in analogy with, conventional methods. The preparation of intermediates and compounds according to the Examples of the present invention may in particular be illuminated by the following Schemes. Definitions of variables in the structures in Schemes herein are commensurate with those of corresponding positions in the formulas delineated herein.

[chemical structure]

Compounds of general formula (I) can easily be prepared from the alcohols of general formula (IV) by either using the alcohol directly or pre-forming the alkoxide using a suitable base/reagent (e.g. NaH) and coupling to a suitably activated A-R¹ or R¹ group (or protected A-R¹ or R¹ group). Activated A-R¹ or R¹ group functionalities typically used for the formation of phosphates, esters, carbonates and carbamates include, but not limited to, phosphoryl chlorides, acid chlorides, activated carboxylic acids, chloroformates, activated carbonates and isocyanates. Alternatively, the A-R¹ or R¹ group can be introduced in a step-wise manner using standard methodologies. Suitable protecting group strategies can be employed where necessary. The formation of (Ia) from (IV) using 2-dimethylaminoethyl carbonochloridate as an activated R group is representative of this approach.

The synthesis of Tonabersat, and other structurally related compounds, is disclosed in WO 95/34545. The present invention encompasses compounds prepared by applying the pro-drug groups -AR¹, R² and Q taught herein to the specific Examples disclosed in WO 95/34545. The methods proposed for the synthesis of compounds of general formula (I) are known to those skilled in the art, for example in Rautio et al., Nature Reviews Drug Discovery, 7, 255-270, 2008.

Optionally, a compound of formula (I) can also be transformed into another compound of formula (I) in one or more synthetic steps.

The following abbreviations have been used:
aq aqueous
Boc tert-butyloxycarbonyl
d day(s)
DCC N,N′-dicyclohexylcarbodiimide
DCM dichloromethane
DIPEA diiisopropylethylamine
DMAP 4-dimethylaminopyridine
DMF dimethylformamide
EDC 1-ethyl-3-(3-dimethylaminopropyl)carbodiimide
ES+ electrospray ionization
Et₃N triethylamine
EtOAc ethyl acetate
Et₂O diethyl ether
EtOH ethanol
h hour(s)
HATU O-(7-azabenzotriazol-1-yl)-N,N,N′,N′-tetramethyluronium hexafluorophosphate
HOBt Hydroxybenzotriazole
HPLC High Performance Liquid Chromatography
LCMS Liquid Chromatography Mass Spectrometry
M molar
MeCN acetonitrile
MeOH methanol
[MH]⁺ protonated molecular ion
min minute(s)
MS Mass Spectrometry
MTBE methyl tert-butyl ether
NMM N-methyl morpholine
PhMe toluene
Rt retention time
sat saturated
tert tertiary
TFA trifluoroacetic acid
THF tetrahydrofuran

### EXAMPLES AND INTERMEDIATE COMPOUNDS

### Experimental Methods

Reactions were conducted at room temperature unless otherwise specified. Preparative chromatography was performed using a Flash Master Personal system equipped with Isolute Flash II silica columns or using a CombiFlash Companion system equipped with GraceResolv silica column. The purest fractions were collected, concentrated and dried under vacuum.

Compounds were typically dried in a vacuum oven at 40° C. prior to purity analysis. Compound analysis was performed by HPLC/LCMS using an Agilent 1100 HPLC system/Waters ZQ mass spectrometer connected to an Agilent 1100 HPLC system with a Phenomenex Synergi, RP-Hydro column (150×4.6 mm, 4 μm, 1.5 mL per min, 30° C., gradient 5-100% MeCN (+0.085% TFA) in water (+0.1% TFA) over 7 min, 200-300 nm). The compounds prepared were named using IUPAC nomenclature.

### Intermediate 1

### tert-Butyl 2-isocyanatoacetate

[chemical structure]

tert-Butyl 2-aminoacetate (7.50 g, 44.7 mmol) was dissolved in DCM (150 mL) and sat aq NaHCO₃ (150 mL), and cooled to 0° C. Triphosgene (4.40 g, 14.8 mmol) was added portion-wise and the reaction mixture was stirred at 0-5° C. for 45 min. The aqueous fraction was extracted with DCM (2×) and the combined organic fractions were dried (MgSO₄) and concentrated in vacuo. The residue was purified by distillation (boiling point 35-37° C./2 mm Hg) to give the title compound (3.41 g, 48.2%) as a colourless liquid.

### Intermediate 2

### tert-Butyl N-[(3S,4S)-6-acetyl-3-hydroxy-2,2-dimethyl-3,4-dihydro-2H-1-benzopyran-4-yl]carbamate

[chemical structure]

1-[(3S,4S)-4-Amino-3-hydroxy-2,2-dimethyl-3,4-dihydro-2H-1-benzopyran-6-yl]ethan-1-one sulfuric acid hydrate (1.20 g, 3.42 mmol) was dissolved in THF (70 mL) and water (6 mL), and 2M aq NaOH (3.40 mL, 6.84 mmol) and Boc₂O (760 mg, 3.48 mmol) were added. The reaction mixture was stirred for 23 h and partitioned between water (180 mL) and EtOAc (120 mL). The aqueous fraction was extracted with EtOAc (120 mL) and the combined organic fractions were dried (MgSO₄) and concentrated in vacuo to give the crude title compound (1.12 g, 97.8%). LCMS (ES⁺): 236.1 [MH-Boc]⁺

### Intermediate 3

### (3S,4S)-6-Acetyl-4-[(3-chloro-4-fluorobenzene)amido]-2,2-dimethyl-3,4-dihydro-2H-1-benzopyran-3-yl 2-aminoacetate hydrochloride

[chemical structure]

N-[(3S,4S)-6-Acetyl-3-hydroxy-2,2-dimethyl-3,4-dihydro-2H-1-benzopyran-4-yl]-3-chloro-4-fluorobenzamide (400 mg, 1.02 mmol) was dissolved in DCM (8 mL) and Boc-Gly-OSu (full name: 2,5-dioxopyrrolidin-1-yl 2-{[(tert-butoxy)carbonyl]amino}acetate) (556 mg, 2.04 mmol), DIPEA (391 μL, 2.25 mmol) and DMAP (12 mg, 0.10 mmol) were added. The reaction mixture was stirred overnight and concentrated in vacuo. The residue was partitioned between EtOAc (15 mL) and 10% aq citric acid solution (10 mL). The organic fraction was washed with water (10 mL) and concentrated in vacuo. The residue was purified by column chromatography on normal phase silica eluting with heptane/EtOAc mixtures. The (3S,4S)-6-acetyl-4-[(3-chloro-4-fluorobenzene)amido]-2,2-dimethyl-3,4-dihydro-2H-1-benzopyran-3-yl 2-{[(tert-butoxy)carbonyl]amino}acetate intermediate was dissolved in 4M HCl in dioxane (4 mL) and stirred for 90 min. The solvents were removed in vacuo and the residue partitioned between EtOAc (10 mL) and sat aq Na₂CO₃ solution (5 mL). The aqueous fraction was extracted with EtOAc (10 mL) and the combined organic fractions were concentrated in vacuo. The residue was purified by column chromatography on normal phase silica eluting with heptane/ethyl acetate mixtures. To each pure fraction was added 1.25M HCl in EtOH (200 μL). The pure fractions were combined and dried in vacuo to give the title compound (93 mg, 18.8%) as a white foam. LCMS (ES⁺): 449.0 [MH]⁺. HPLC: Rt 4.95 min, 96.9% purity.

### Intermediate 4

### (3S,4S)-6-Acetyl-4-[(3-chloro-4-fluorobenzene)amido]-2-dimethyl-3,4-dihydro-2H-1-benzopyran-3-yl (2S)-2-amino-3-methylbutanoate hydrochloride

[chemical structure]

Boc-Val-OH (full name: (2S)-2-{[(tert-butoxy)carbonyl]amino}-3-methylbutanoic acid) (521 mg, 2.40 mmol), EDC.HCl (537 mg, 2.80 mmol), HOBt (429 mg, 2.80 mmol) and DMAP (733 mg, 6.00 mmol) were dissolved in DCM (15 mL) and the reaction mixture was stirred for 15 min. N-[(3S,4S)-6-Acetyl-3-hydroxy-2,2-dimethyl-3,4-dihydro-2H-1-benzopyran-4-yl]-3-chloro-4-fluorobenzamide (784 mg, 2.00 mmol) was added and the reaction mixture was stirred overnight. The reaction mixture was washed with 10% aq citric acid, water, 10% aq NaHCO₃ and brine, dried (MgSO₄) and concentrated in vacuo to give crude intermediate (3S,4S)-6-acetyl-4-[(3-chloro-4-fluorobenzene)amido]-2,2-dimethyl-3,4-dihydro-2H-1-benzopyran-3-yl (2S)-2-{[(tert-butoxy)carbonyl]amino}-3-methylbutanoate (1.20 g). This material (1.20 g) was dissolved in MeOH (2 mL) and 4M HCl in dioxane (20 mL) and the reaction mixture was stirred for 1 h and concentrated in vacuo. The residue was triturated from MTBE, washed with hexane and purified by column chromatography on normal phase silica eluting with DCM/MeOH/NH₄OH (100:2.5:0.5). The residue was dissolved in Et₂O and 2M HCl in Et₂O was added. The resulting precipitate was collected by filtration and washed with Et₂O and hexane to give the title compound (172 mg, 16.3%) as a white solid. LCMS (ES⁺): 491.1 [MH]⁺. HPLC: Rt 5.38 min, 98.1% purity.

### Example 1

### (3S,4S)-6-Acetyl-4-[(3-chloro-fluorobenzene)amido]-2,2-dimethyl-3,4-dihydro-2H-1-benzopyran-3-yl 2-(dimethylamino)ethyl carbonate

[chemical structure]

Triphosgene (198 mg, 0.67 mmol) was dissolved in DCM (10 mL) and a solution of 2-dimethylaminoethanol (201 uL, 2.00 mmol) and DMAP (244 mg, 2.00 mmol) in DCM (10 mL) was added. The reaction mixture was stirred for 4 h. A solution of N-[(3S,4S)-6-acetyl-3-hydroxy-2,2-dimethyl-3,4-dihydro-2H-1-benzopyran-4-yl]-3-chloro-4-fluorobenzamide (784 mg, 2.00 mmol) and DMAP (488 mg, 4.00 mmol) in DCM (10 mL) was added and the reaction mixture was stirred overnight. The solution was absorbed onto silica and purified by column chromatography on normal phase silica eluting with EtOAc to give the title compound (308 mg, 30.4%) as a white solid. LCMS (ES⁺): 507.0 [MH]⁺. HPLC: Rt 5.27 min, 97.5% purity.

### Example 2

### (3S,4S)-6-Acetyl-4-[(3-chloro-4-fluorobenzene)amido]-2,2-dimethyl-3,4-dihydro-2H-1-benzopyran-3-yl 2-(trimethylazaniumyl)ethyl carbonate iodide

[chemical structure]

EXAMPLE 1 (150 mg, 0.30 mmol) was dissolved in Et₂O/DCM (16 mL, 3:1) and iodomethane (300 uL, 4.82 mmol) was added. The reaction mixture was allowed to stand over the weekend and the resulting precipitate was collected by filtration and washed with Et₂O to give the title compound (109 mg, 56.9%) as an off-white solid, in two batches. LCMS (ES⁺): 521.1 [M]⁺. HPLC: Rt 5.37 min, 99.0% purity.

### Example 3

### (3S,4S)-6-Acetyl-4-(3-chloro-4-fluorobenzene)amido-2,2-dimethyl-3,4-dihydro-2H-1-benzopyran-3-yl 2-(diethylamino)ethyl carbonate

[chemical structure]

2-Diethylaminoethanol (1.30 g, 11.1 mmol) was dissolved in Et₂O (50 mL) and added drop-wise at 0° C. to a solution of 4-nitrophenylchloroformate (2.24 g, 11.1 mmol) in Et₂O (40 mL). The reaction mixture was stirred over the weekend and the resulting precipitate was collected by filtration and washed with Et₂O to give a white solid (2.90 g, 82.2%). The 2-diethylamino)ethyl 4-nitrophenyl carbonate hydrochloride intermediate (1.43 g, 4.50 mmol) and N-[(3S,4S)-6-acetyl-3-hydroxy-2,2-dimethyl-3,4-dihydro-2H-1-benzopyran-4-yl]-3-chloro-4-fluorobenzamide (1.18 g, 3.00 mmol) were dissolved in DCM (50 mL), DMAP (1.10 g, 9.00 mmol) was added and the reaction mixture was stirred overnight. The reaction mixture was washed with 2% aq NaOH (2×50 mL), sat aq NaHCO₃ (3×50 mL), dried (MgSO₄), absorbed onto silica and purified by column chromatography on normal phase silica eluting with pentane/EtOAc (1:1) then EtOAc to give the title compound (360 mg, 22.4%) as a white solid. LCMS (ES⁺): 535.1 [MH]⁺. HPLC: Rt 5.60 min, 97.6% purity.

### Example 4

### (3S,4S)-Acetyl-4-[(3-chloro-4-fluorobenzene)amido]-2,2-dimethyl-3,4-dihydro-2H-1-benzopyran-3-yl 2-(morpholin-4-yl)ethyl carbonate

[chemical structure]

2-Hydroxyethylmorpholine (727 mg, 5.54 mmol) was dissolved in Et₂O (20 mL) and added drop-wise at 0° C. to a solution of 4-nitrophenylchloroformate (1.17 g, 5.81 mmol) in Et₂O (25 mL). The reaction mixture was stirred overnight and the resulting precipitate was collected by filtration and washed with Et₂O to give a white solid (1.84 g, 91.8%). The 2-(morpholin-4-yl)ethyl 4-nitrophenyl carbonate hydrochloride intermediate (998 mg, 3.00 mmol) and N-[(3S,4S)-8-acetyl-3-hydroxy-2,2-dimethyl-3,4-dihydro-2H-1-benzopyran-4-yl]-3-chloro-4-fluorobenzamide (1.18 g, 3.00 mmol) were dissolved in DCM (50 mL), DMAP (806 mg, 6.60 mmol) was added and the reaction mixture was stirred over the weekend. The reaction mixture was absorbed onto silica and purified by column chromatography on normal phase silica eluting with hexane/EtOAc (1:1) then EtOAc. The residue was dissolved in EtOAc, washed with 2% aq NaOH (2×50 mL), sat aq NaHCO₃ (3×50 mL), dried (MgSO₄) and concentrated in vacuo. The residue was triturated from hexane (50 mL) and washed with pentane to give the title compound (558 mg, 33.9%) as a white solid. LCMS (ES⁺): 549.1 [MH]⁺. HPLC: Rt 5.42 min, 98.8% purity.

### Example 5

### (2)-3-[({[(3S,4S)-Acetyl-4-[(3-chloro-4-fluorobenzene)amido]-2,2-dimethyl-3,4-dihydro-2H-1-benzopyran-3-yl]oxy}carbonyl)oxy]-2-aminopropanoic acid hydrochloride

[chemical structure]

N-[(3S,4S)-6-Acetyl-3-hydroxy-2,2-dimethyl-3,4-dihydro-2H-1-benzopyran-4-yl]-3-chloro-4-fluorobenzamide (2.64 g, 6.74 mmol) and pyridine (1.20 mL, 14.8 mmol) were dissolved in DCM (50 mL) and triphosgene (669 mg, 2.23 mmol) was added. The reaction mixture was stirred for 1 h and a solution of tert-butyl (2S)-2-([(tert-butoxy)carbonyl]amino-3-hydroxypropanoate (1.76 g, 6.74 mmol) in DCM (30 mL) was added. The reaction mixture was stirred for 19 h, diluted with water (70 mL) and extracted into DCM (70 mL), dried (MgSO₄) and concentrated in vacuo. The residue was purified by column chromatography on normal phase silica eluting with hexane/EtOAc (2:1) to give intermediate tert-butyl (2S)-3-[({[(3S,4S)-6-acetyl-4-[(3-chloro-4-fluorobenzene)amido]-2,2-dimethyl-3,4-dihydro-2H-1-benzopyran-3-yl]oxy}carbonyl)oxy]-2-{[(tert-butoxy)carbonyl]amino}propanoate (4.58 g, 68.8%). This material (1.50 g, 2.21 mmol) was dissolved in DCM, 4M HCl in dioxane (15 mL) was added and the reaction mixture was stirred for 2 d. The reaction mixture was concentrated in vacuo and the residue was triturated from hexane (40 mL). The residue was suspended in Et₂O (10 mL) and stirred overnight. The resulting precipitate was collected by filtration to give the title compound (1.06 g, 85.8%) as a cream solid. LCMS (ES⁺): 523.0 [MH]⁺. HPLC: Rt 4.92 min, 94.3% purity.

### Example 6

### Sodium 2-[({[(3S,4S)-6-acetyl-4-[(3-chloro-4-fluorobenzene)amido]-2,2-dimethyl-3,4-dihydro-2H-1-benzopyran-3-yl]oxy}carbonyl)oxy]ethane-1-sulfonate

[chemical structure]

N-[(3S,4S)-6-Acetyl-3-hydroxy-2,2-dimethyl-3,4-dihydro-2H-1-benzopyran-4-yl]-3-chloro-4-fluorobenzamide (3.92 g, 10.0 mmol) and pyridine (2.5 mL) were dissolved in DCM (125 mL) and triphosgene (980 mg, 3.33 mmol) was added. The reaction mixture was stirred for 2 h. Sodium isethionate (1.48 g, 10.0 mmol) was added and the reaction mixture was stirred overnight. The reaction mixture was diluted with DCM (50 mL) and EtOAc (100 mL) and washed with brine, dried (MgSO4) and concentrated in vacuo. The residue was dissolved in EtOAc (50 mL), filtered and passed through a plug of silica. The residue was triturated from diisopropyl ether then hexane to give the title compound (133 mg, 2.3%) as a cream solid. LCMS (ES⁺): 544.0 [MH]⁺.

### Example 7

### 2-[({[(3S,4S)-6-Acetyl-4-[(3-chloro-4-fluorobenzene)amido]-2,2-dimethyl-3,4-dihydro-2H-1-benzopyran-3-yl]oxy}carbonyl)amino]acetic acid

[chemical structure]

N-[(3S,4S)-6-Acetyl-3-hydroxy-2,2-dimethyl-3,4-dihydro-2H-1-benzopyran-4-yl]-3-chloro-4-fluorobenzamide (392 mg, 1.00 mmol) and Et₃N (349 uL, 2.50 mmol) were dissolved in PhMe (7 mL), Intermediate 1 (393 mg, 2.50 mmol) was added and the reaction mixture was heated under reflux for 4 h. Further Intermediate 1 (100 mg, 0.64 mmol) was added and the reaction mixture was heated under reflux overnight. The reaction mixture was concentrated in vacuo. The reaction was similarly repeated on 2.5 times scale and the combined residues were purified by column chromatography on normal phase silica eluting with hexane/EtOAc (2:1) and triturated from hexane to give a white solid (1.50 g, 78%). The tert-butyl 2-[({[(3S,4S)-6-acetyl-4-[(3-chloro-4-fluorobenzene)amido]-2,2-dimethyl-3,4-dihydro-2H-1-benzopyran-3-yl]oxy}carbonyl)amino]acetate intermediate (1.50 g, 2.73 mmol) was dissolved in DCM (6 mL), cooled to 0° C. and TFA (6 mL) was added. The reaction mixture was stirred overnight and concentrated in vacuo. The residue was dissolved in EtOAc and extracted into 1M aq NaOH. The aqueous fraction was acidified with 1M aq HCl and extracted into EtOAc, washed with brine, dried (MgSO₄) and concentrated in in vacuo. The residue was purified by column chromatography on normal phase silica eluting with DCM/MeOH/Et₃N (100:5:1), dissolved in water and acidified with 1M aq HCl. The resulting precipitate was collected by filtration and washed with water to give the title compound (680 mg, 50%) as a white solid. LCMS (ES⁺): 493.1 [MH]⁺. HPLC: Rt 5.54 min, 100% purity.

### Example 8

### (2S)-2-[({[(3S,4S)-6-Acetyl-4-[(3-chloro-4-fluorobenzene)amido]-2,2-dimethyl-3,4-dihydro-2H-1-benzopyran-3-yl]oxy}carbonyl)amino]-3-methylbutanoic acid

[chemical structure]

N-[(3S,4S)-6-Acetyl-3-hydroxy-2,2-dimethyl-3,4-dihydro-2H-1-benzopyran-4-yl]-3-chloro-4-fluorobenzamide (784 mg, 2.00 mmol) and pyridine (440 uL, 5.44 mmol) were dissolved in DCM (15 mL) and the reaction mixture was cooled to 0° C. Triphosgene (196 mg, 0.66 mmol) was added and the reaction mixture was stirred for 1 h. A solution of L-valine tert-butyl ester hydrochloride (419 mg, 2.00 mmol) in DCM (10 mL) was added at 0° C. and the reaction mixture was stirred overnight. The reaction mixture was washed with 2M aq HCl, dried (MgSO₄), absorbed onto silica and purified by column chromatography on normal phase silica eluting with hexane/EtOAc (3:1) to give a white solid (581 mg, 47.5%). The tert-butyl (2S)-2-[({[(3S,4S)-6-acetyl-4-[(3-chloro-4-fluorobenzene)amido]-2,2-dimethyl-3,4-dihydro-2H-1-benzopyran-3-yl]oxy}carbonyl)amino]-3-methylbutanoate intermediate (561 mg, 0.95 mmol) was dissolved in 4M HCl in dioxane (10 mL), stirred over the weekend and concentrated in vacuo. The residue was dissolved in DCM and washed with 1M aq NaOH (100 mL). The organic fraction was acidified with 2M aq HCl and extracted into EtOAc (50 mL×4), dried (MgSO₄) and concentrated in vacuo. The residue was purified by column chromatography on normal phase silica eluting with hexane/EtOAc (1:1) and trituration from pentane/diisopropyl ether (10:1). The residue was dissolved in water and acidified with 1M aq HCl and the resulting precipitate was collected by filtration and washed with water to give the title compound (158 mg, 31.1%) as a pale green solid. LCMS (ES⁺): 535.0 [MH]⁺. HPLC: Rt 6.04 min, 100% purity.

### Example 9

### (2S)-2-[({[(3S,4S)-6-acetyl-4-[(3-chloro-4-fluorobenzene)amido]-2,2-dimethyl-3,4-dihydro-2H-1-benzopyran-3-yl]oxy}carbonyl)amino]-6-aminohexanoic acid hydrochloride

[chemical structure]

N-[(3S,4S)-6-Acetyl-3-hydroxy-2,2-dimethyl-3,4-dihydro-2H-1-benzopyran-4-yl]-3-chloro-4-fluorobenzamide (784 mg, 2.00 mmol) and pyridine (440 uL, 5.44 mmol) were dissolved in DCM (15 mL), triphosgene (196 mg, 0.66 mmol) was added and the reaction mixture was stirred for 1 h.

A solution of (2S)-2-amino-6-{[(tert-butoxy)carbonyl]amino}hexanoic acid hydrochloride (678 mg, 2.00 mmol) in DCM (10 mL) was added and the reaction mixture was stirred overnight. Water (20 mL) was added and the aqueous fraction was extracted with DCM (20 mL). The combined organic fractions were dried (MgSO₄), concentrated in vacuo and purified by column chromatography on normal phase silica eluting with hexane/EtOAc (1:1) to give intermediate tert-butyl (2S)-2-[({[(3S,4S)-6-acetyl-4-[(3-chloro-4-fluorobenzene)amido]-2,2-dimethyl-3,4-dihydro-2H-1-benzopyran-3-yl]oxy}carbonyl)amino]-6-{[(tert-butoxy)carbonyl]amino}hexanoate (1.21 g, 84.0%). This material (990 mg, 1.37 mmol) was dissolved in TFA (4 mL) and stirred for 6 h. The reaction mixture was partitioned between EtOAc (25 mL) and water (40 mL) and the aqueous fraction was extracted with EtOAc (25 mL). The combined organic fractions were dried (MgSO₄) and concentrated in vacuo to give the title compound (912 mg, 96.0%) as an off white solid. LCMS (ES⁺): 564.0 [MH]⁺. HPLC: Rt 4.85 min, 93.2% purity.

### Example 10

### (3S,4S)-6-Acetyl-4-[(3-chloro-4-fluorobenzene)amido]-2,2-dimethyl-3,4-dihydro-2H-1-benzopyran-3-yl 2-[(2S)-pyrrolidin-2-ylformamido]acetate

[chemical structure]

N-[(3S,4S)-8-Acetyl-3-hydroxy-2,2-dimethyl-3,4-dihydro-2H-1-benzopyran-4-yl]-3-chloro-4-fluorobenzamide (784 mg, 2.00 mmol), DMAP (20 mg, 0.16 mmol) and 2-{[(2S)-1-[(tert-butoxy)carbonyl]pyrrolidin-2-yl]formamido}acetic acid (544 mg, 2.00 mmol) were dissolved in DCM (10 mL), a solution of DCC (619 mg, 3.05 mmol) in DCM (10 mL) was added and the reaction mixture was stirred for 3 h. The reaction mixture was filtered and the filtrate was concentrated in vacuo and purified by column chromatography on normal phase silica eluting with hexane/EtOAc (1:1) to give intermediate tert-butyl (2S)-2-[(2-([(3S,4S)-6-acetyl-4-[(3-chloro-4-fluorobenzene)amido]-2,2-dimethyl-3,4-dihydro-2H-1-benzopyran-3-yl]oxy)-2-oxoethyl)carbamoyl]pyrrolidine-1-carboxylate (886 mg, 68.6%). This material (886 mg, 1.36 mmol) was dissolved in DCM (4 mL), 4M HCl in dioxane (10 mL) was added and the reaction mixture was stirred for 3 h and concentrated in vacuo (930 mg crude residue). 390 mg of the residue was triturated from hexane (10 mL) to give the title compound (265 mg, 79.6%) as a white solid. LCMS (ES⁺): 546.1 [MH]⁺. HPLC: Rt 5.14 min, 96.5% purity.

### Example 11

### (3S,4S)-6-Acetyl-4-[(3-chloro-4-fluorobenzene)amido]-2,2-dimethyl-3,4-dihydro-2H-1-benzopyran-3-yl 2-[(2S)-2-amino-4-methylpentanamido]acetate hydrochloride

[chemical structure]

Boc-Leu-OH (full name: (2S)-2-{[(tert-butoxy)carbonyl]amino}-4-methylpentanoic acid) (483 mg, 2.00 mmol) and HATU (913 mg, 2.40 mmol) were dissolved in DCM (20 mL) and DMF (2 mL) and the reaction mixture was stirred for 30 min. Intermediate 3 (971 mg, 2.00 mmol) and NMM (607 mg, 6.00 mmol) were added and the reaction mixture was stirred for 5 h and concentrated in vacuo. The residue was dissolved in EtOAc and washed with 10% aq citric acid. The organic fraction was washed with brine, dried (MgSO₄) and concentrated in vacuo. The residue was purified by column chromatography on normal phase silica eluting with hexane/EtOAc (65:35) to give intermediate (3S,4S)-6-acetyl-4-[(3-chloro-4-fluorobenzene)amido]-2,2-dimethyl-3,4-dihydro-2H-1-benzopyran-3-yl 2-[(2S)-2-{[(tert-butoxy)carbonyl]amino}-4-methylpentanamido]acetate (1.07 g, 80.8%). This material (1.00 g, 1.51 mmol) was dissolved in MeOH (15 mL), 4M HCl in dioxane (15 mL) was added and the reaction mixture was stirred for 3.5 h and concentrated in vacuo. The residue was triturated from hexane and washed with Et₂O and hexane to give the title compound (758 mg, 83.8%) as an off white solid. LCMS (ES⁺): 562.0 [MH]⁺. HPLC: Rt 5.38 min, 97.5% purity.

### Example 12

### (3S,4S)-6-Acetyl-4-[(3-chloro-4-fluorobenzene)amido]-2,2-dimethyl-3,4-dihydro-2H-1-benzopyran-3-yl 2-(2-aminoacetamido)acetate hydrochloride

[chemical structure]

Boc-Gly-OH (full name: 2-{[(tert-butoxy)carbonyl]amino}acetic acid) (389 mg, 2.22 mmol), EDC.HCl (511 mg, 2.67 mmol) and HOBt (409 mg, 2.67 mmol) were dissolved in DCM (20 mL), Intermediate 3 (1.08 g, 2.22 mmol) and DIPEA (1.42 mL, 8.19 mmol) were added and the reaction mixture was stirred over the weekend. The reaction mixture was diluted with DCM, washed with 2M aq HCl and sat aq NaHCO₃, dried (MgSO₄) and concentrated in vacuo. The residue was purified by column chromatography on normal phase silica eluting with hexane/EtOAc (2:1 then 1:1) to give intermediate (3S,4S)-6-acetyl-4-[(3-chloro-4-fluorobenzene)amido]-2,2-dimethyl-3,4-dihydro-2H-1-benzopyran-3-yl 2-(2-{[(tert-butoxy)carbonyl]amino}acetamido)acetate (887 mg, 65.8%). This material (887 mg, 1.46 mmol) was dissolved 4M HCl in dioxane (20 mL) and the reaction mixture was stirred for 1 h and concentrated in vacuo. The residue was partitioned between EtOAc and 1M aq NaOH and the organic fraction was dried (MgSO₄) and concentrated in vacuo. The residue was dissolved in Et₂O and EtOAc, and 2M HCl in Et₂O (4 mL) was added. The resulting precipitate was collected by filtration and washed with Et₂O to give the title compound (529 mg, 66.6%) as a beige solid. LCMS (ES⁺): 506.0 [MH]⁺. HPLC: Rt 4.91 min, 96.9% purity.

### Example 13

### (3S,4S)-6-Acetyl-4-[(3-chloro-4-fluorobenzene)amido]-2,2-dimethyl-3,4-dihydro-2H-1-benzopyran-3-yl 2-[(2S,3S)-2-amino-3-methylpentanamido]acetate hydrochloride

[chemical structure]

Boc-Ile-OH (full name: (2S,3S)-2-{[(tert-butoxy)carbonyl]amino}-3-methylpentanoic acid) (477 mg, 2.06 mmol), EDC.HCl (474 mg, 2.47 mmol) and HOBt (379 mg, 2.47 mmol) were dissolved in DCM (20 mL) and the reaction mixture was cooled to 0° C. Intermediate 3 (1.00 g, 2.06 mmol) and DIPEA (1.32 mL, 7.60 mmol) were added and the reaction mixture was stirred overnight. The reaction mixture was diluted with DCM (30 mL), washed with 2M aq HCl (50 mL) and sat aq NaHCO₃ (50 mL), dried (MgSO₄) and concentrated in vacuo. The residue was purified by column chromatography on normal phase silica eluting with hexane/EtOAc (2:1) to give intermediate (3S,4S)-6-acetyl-4-[(3-chloro-4-fluorobenzene)amido]-2,2-dimethyl-3,4-dihydro-2H-1-benzopyran-3-yl 2-[(2S,3S)-2-{[(tert-butoxy)carbonyl]amino}-3-methylpentanamido]acetate (1.36 g, 56.9%). This material (777 mg, 1.17 mmol) was dissolved in 4M HCl in dioxane (10 mL) and the reaction mixture was stirred overnight and concentrated in vacuo. The residue was partitioned between EtOAc and 1M aq NaOH and the organic fraction was dried (MgSO₄) and concentrated in vacuo. The residue was dissolved in Et₂O and 2M HCl in Et₂O was added. The resulting precipitate was collected by filtration and washed with Et₂O to give the title compound (537 mg, 76.5%) as a beige solid. LCMS (ES⁺): 562.1 [MH]⁺. HPLC: Rt 5.61 min, 96.1% purity.

### Example 14

### (3S,4S)-6-Acetyl-4-[(3-chloro-4-fluorobenzene)amido]-2,2-dimethyl-3,4-dihydro-2H-1-benzopyran-3-yl 2-[(2S)-2-amino-3-methylbutanamido]acetate hydrochloride

[chemical structure]

Boc-L-Valine hydroxysuccinimide ester (408 mg, 1.30 mmol), Intermediate 3 (350 mg, 0.72 mmol) and DIPEA (553 uL, 3.17 mmol) were dissolved in DCM (25 mL) and the reaction mixture was stirred for 20 h, diluted with DCM (10 mL) and washed with sat aq NH₄Cl (2×25 mL). The organic fraction was dried (MgSO₄) and concentrated in vacuo. The residue was purified by column chromatography, dissolved in 1M HCl (20 mL) and stirred overnight. The reaction mixture was concentrated in vacuo and the residue was purified by column chromatography to give the title compound as an off white solid (262 mg, 62.1%). LCMS (ES⁺): 548.0 [MH]⁺. HPLC: Rt 5.20 min, 98.0% purity.

### Example 15

### (3S,4S)-4-Acyl-4-[(3-chloro-4-fluorobenzene)amido]-2,2-dimethyl-3,4-dihydro-2H-1-benzopyran-3-yl (2S)-2-[(2S)-2-amino-3-methylbutanamido]-3-methylbutanoate hydrochloride

[chemical structure]

Boc-L-Valine hydroxysuccinimide ester (220 mg, 0.70 mmol), Intermediate 4 (307 mg, 0.58 mmol) and DIPEA (446 uL, 2.56 mmol) were dissolved in DCM (25 mL) and the reaction mixture was stirred overnight, diluted with DCM (10 mL) and washed with sat aq NH₄Cl (2×25 mL). The organic fraction was dried (MgSO₄) and concentrated in vacuo. The residue was purified by column chromatography, dissolved in 1M HCl (20 mL) and stirred overnight. The reaction mixture was concentrated in vacuo and the residue was purified by column chromatography to give the title compound as a white solid (129 mg, 35.4%). LCMS (ES⁺): 590.1 [MH]⁺. HPLC: Rt 5.85 min, 99.4% purity.

### Example 16

### (3S,4S)-6-Acetyl-4-[(3-chloro-4-fluorobenzene)amido]-2,2-dihydro-3,4-dihydro-2H-1-benzopyran-3-yl (2S)-2-(2-aminoacetamido)-3-methylbutanoate hydrochloride

[chemical structure]

Boc-Gly-OH (full name: 2-{[(tert-butoxy)carbonyl]amino}acetic acid) (164 mg, 0.95 mmol), EDC.HCl (218 mg, 1.14 mmol) and HOBt (174 mg, 1.14 mmol) were dissolved in DCM (10 mL) and the reaction mixture was cooled to 0° C. Intermediate 4 (500 mg, 0.95 mmol) and DIPEA (0.6 mL, 3.45 mmol) were added and the reaction mixture was stirred overnight. The reaction mixture was diluted with DCM, washed with 2M aq HCl and sat aq NaHCO₃, dried (MgSO₄) and concentrated in vacuo to give crude intermediate (3S,4S)-acetyl-4-[(3-chloro-4-fluorobenzene)amido]-2,2-dimethyl-3,4-dihydro-2H-1-benzopyran-3-yl (2S)-2-(2-{[(tert-butoxy)carbonyl]amino}acetamido)-3-methylbutanoate (614 mg, 82.3%). This material (506 mg, 0.78 mmol) was dissolved in 4M HCl in dioxane (10 mL) and the reaction mixture was stirred for 1.5 h and concentrated in vacuo. The residue was triturated from MTBE and washed with MTBE. The residue was suspended in Et₂O and the reaction mixture was stirred for 1 h. The precipitate was collected by filtration and washed with Et₂O. The residue was partitioned between EtOAc and 1M aq NaOH and the organic fraction was dried (MgSO₄) and concentrated in vacuo. The residue was dissolved in Et₂O and 2M HCl in Et₂O was added. The resulting precipitate was collected by filtration and washed with Et₂O to give the title compound (191 mg, 42.0%) as a pale yellow solid. LCMS (ES⁺): 548.0 [MH]⁺. HPLC: Rt 5.33 min, 92.5% purity.

### Example 17

### (3S,4S)-6-Acetyl-4-[(3-chloro-4-fluorobenzene)amido]-2,2-dimethyl-3,4-dihydro-2H-1-benzopyran-3-yl 4-(morpholin-4-yl)butanoate hydrochloride

[chemical structure]

N-[(3S,4S)-6-Acetyl-3-hydroxy-2,2-dimethyl-3,4-dihydro-2H-1-benzopyran-4-yl]-3-chloro-4-fluorobenzamide (1.33 g, 3.39 mmol), 4-(morpholin-4-yl)butanoic acid (1.00 g, 4.77 mmol) and DMAP (41.0 mg, 0.34 mmol) were dissolved in DCM (40 mL) and the reaction mixture was cooled to 0° C. and a 1M solution of DCC in DCM (5.1 mL) was added drop-wise. The reaction mixture was stirred for 4 h and filtered and the filtrate was concentrated in vacuo. The residue was purified by column chromatography on normal phase silica eluting with DCM/MeOH/NH₄OH (100/2.5/0.5). The residue was dissolved in Et₂O and 2M HCl in Et₂O was added. The resulting precipitate was collected by filtration and washed with Et₂O. The residue was dissolved in water, filtered and the filtrate washed with ether, basified with 10% aq NaHCO₃ and extracted into EtOAc. The organic fraction was washed with brine, dried (MgSO₄) and concentrated in vacuo. The residue was dissolved in Et₂O and 2M HCl in Et₂O was added. The resulting precipitate was collected by filtration and washed with Et₂O to give the title compound (1.00 g, 50.5%) as a white solid. LCMS (ES⁺): 547.2 [MH]⁺. HPLC: Rt 5.29 min, 95.1% purity.

### Example 18

### (3S,4S)-6-Acetyl-4-[(3-chloro-4-fluorobenzene)amido]-2,2-dimethyl-3,4-dihydro-2H-1-benzopyran-3-yl 4-(piperidin-1-yl)piperidine-1-carboxylate hydrochloride

[chemical structure]

N-[(3S,4S)-6-Acetyl-3-hydroxy-2,2-dimethyl-3,4-dihydro-2H-1-benzopyran-4-yl]-3-chloro-4-fluorobenzamide (784 mg, 2.00 mmol) was dissolved in THF (25 mL) and the reaction mixture was cooled to 0° C. NaH (100 mg, 60% dispersion in mineral oil, 2.50 mmol) was added and the reaction mixture was stirred at 0° C. for 20 min. 4-Piperidinopiperidine-1-carbonyl chloride (461 mg, 2.00 mmol) was added portion-wise and the reaction mixture was stirred overnight and concentrated in vacuo. The residue was partitioned between 15% aq NH₄Cl and EtOAc and the organic fraction was washed with brine, dried (MgSO₄) and concentrated hi vacuo. The residue was purified by column chromatography on normal phase silica eluting with DCM/MeOH/NH₄OH (100/2.5/0.5) and dissolved in Et₂O and 2M HCl in Et₂O was added. The resulting precipitate was collected by filtration and washed with Et₂O to give the title compound (900 mg, 72.3%) as a white solid. LCMS (ES⁺): 586.2 [MH]⁺. HPLC: Rt 5.41 min, 99.8% purity.

### Example 19

### (3S,4S)-6-Acetyl-4-amino-2,2-dimethyl-3,4-dihydro-2H-1-benzopyran-3-yl 3-chloro-4-fluorobenzoate hydrochloride

[chemical structure]

Intermediate 2 (566 mg, 1.69 mmo), DMAP (20.0 mg, 0.16 mmol) and 3-chloro-4-fluorobenzoic acid (295 mg, 1.69 mmol) were dissolved in DCM (10 mL) and a solution of DCC (522 mg, 2.53 mmol) in DCM (10 mL) was added. The reaction mixture was stirred overnight and purified by column chromatography on normal phase silica eluting with hexane/EtOAc (3:1) to give the title compound (784 mg, 94.4%). The (3S,4S)-6-acetyl-4-{[(tert-butoxy)carbonyl]amino}-2,2-dimethyl-3,4-dihydro-2H-1-benzopyran-3-yl 3-chloro-4-fluorobenzoate intermediate (784 mg, 1.59 mmol) was dissolved in DCM (6 mL) and 4M HCl in dioxane (4 mL) was added. The reaction mixture was stirred for 20 h and concentrated in vacuo. The residue was suspended in hexane (40 mL), stirred for 1 h and the resulting precipitate was collected by filtration to give the title compound (174 mg, 25.5%) as an off white solid. LCMS (ES⁺): 392.0 [MH]⁺. HPLC: Rt 5.44 min, 95.8% purity.

### Preparation of Compounds of Formula (II)

[chemical structure]

[chemical structure]

Compounds of general formula (IIa) can easily be prepared from the alcohols of general formula (IVa) by protecting the hydroxyl functionality with a suitable protecting group P² to give compounds of general formula (VI) and then coupling the prodrug functionality onto the amide nitrogen atom in one or more steps using synthetic strategies analogous to those used for the synthesis of compounds of general formula (I). The final step is to remove the protecting group P² to give compounds of general formula (IIa).

### Preparation of Compounds of Formula (IIIa) and (IIIb)

[chemical structure]

[chemical structure]

Compounds of general formula (IIIa) can easily be prepared from the ketones of general formula (Id) by either using an alcohol or diol in the presence of an acid and removal of the water generated to prepare acyclic or cyclic ketals respectively. Such methods proposed for the synthesis of compounds of general formula (IIa) are known to those skilled in the art, for example in T. W. Greene & P. G. M. Wuts, Protective Groups in Organic Synthesis (2nd edition) J. Wiley & Sons, 1991 and P. J. Kocienski, Protecting Groups, Georg Thieme Verlag, 1994.

[chemical structure]

Compounds of general formula (IIIb) can easily be prepared from the ketones of general formula (I) where Q=O by using the appropriate hydroxylamine and removal of the water generated to prepare the ketoxime. Such methods proposed for the synthesis of compounds of general formula (IIIb) are known to those skilled in the art, for example in T. W. Greene & P. G. M. Wuts, Protective Groups in Organic Synthesis (2nd edition) J. Wiley & Sons, 1991

Biological Rational

Without wishing to be bound by theory, the general mode of action of the claimed pro-drugs is as follows. For IV administration the high solubility conferred by the solubilising pro-moiety to the parent Tonabersat-like drug is expected to allow a rapid bolus injection whereupon the pro-drug will be quickly cleaved by plasma esterases/phosphatases to reveal the parent drug. For PO administration the mode of action is either where the solubilising pro-drug is predominantly cleaved in the gut by esterases/phosphatases prior to absorption of the parent drug into the systemic circulation, or where the solubilising pro-drug is absorbed intact and then quickly cleaved by plasma esterases/phosphatases to reveal the parent drug.

Solubility

In an embodiment prodrugs of the present invention are suitable for oral administration. The skilled person understands that the pH of the gastrointestinal tract changes along its length. For example, the stomach has a pH of around pH 1.5 and the GI tract after the stomach has a pH of around 5 to 7.5. For more detail see, for example, Measurement of gastrointestinal pH profiles in normal ambulant human subjects, Gut. 1988 August; 29(8): 1035-1041. Improved solubility is expected to result in improved absorption, and therefore improved oral bioavailability. Thus improved solubility at any pH value between around pH 1.5 to 8 is expected to improve oral bioavailability. Compounds of the invention were assessed for solubility in aqueous solutions having a pH of from 2 to 10. In an embodiment prodrugs of the Invention have a solubility of >0.5 mg/mL in an aqueous solution having a pH of from 2 to 8. In an embodiment prodrugs have a solubility of >5.0 mg/mL, or >10.0 mg/mL, >100.0 mg/mL, or >200.0 mg/mL. In an embodiment the prodrugs have the aforementioned aqueous solubility at a pH within the range of from 4 to 8, or from 6 to 8.

In an embodiment prodrugs of the invention are administered intravenously. High prodrug solubility is advantageous in order to reduce the volume of solution administered to the patient, and to reduce the risk of damage to the circulatory system. Solubility of >10 mg/mL is preferred. Yet more preferred is solubility of >30 mg/mL or >100.0 mg/mL. Yet more preferred is solubility of >200.0 mg/mL. The solubility is measured in an aqueous solution having a pH of from 2 to 10, which pH range is advantageous for intravenous prodrug delivery. See, for example, A guide on intravenous drug compatibilities based on their pH, Nasser S C et al./Pharmacie Globale (IJCP) 2010, 5 (01)). In an embodiment the prodrugs of the claimed invention have solubility of >10 mg/mL in an aqueous solution having a pH of from 2 to 10.

Pharmacokinetics

Example Prodrugs of the claimed invention were dosed either intravenously or orally to fasted male Sprague Dawley rats. The rats underwent surgery for jugular vein cannulation 48 h prior to dosing. Following dosing, 0.25 mL blood samples were taken via the cannulae at 0, 5, 10, 20, 30, 45, 60, 120, 240 & 360 min in EDTA coated tubes. Tubes were spun at 13,000 rpm for 4 min and 100 ul of supernatant taken immediately and stored at −80° C. prior to analysis. Plasma samples were analysed by LC-MS/MS following extraction by protein precipitation, and levels of parent prodrug and tonabersat were measured by MRM (Multiple Reaction Monitoring) analysis against an extracted calibration curve of plasma samples spiked with the Example prodrug and tonabersat.

The exposure of tonabersat in plasma following dosing of the prodrugs of the invention was compared directly to the exposure observed following dosing of an equimolar amount of tonabersat under analogous assay conditions (5.00 mg/kg oral dosing or 0.78 mg/kg intravenous dosing). In an embodiment prodrugs of the present invention have >40% exposure of tonabersat obtained following either oral or intravenous dosing of the prodrug, compared to the exposure obtained from dosing an equimolar amount of tonabersat itself. In an embodiment the exposure of tonabersat following dosing of the prodrugs is >50% or preferably >70% compared to the exposure obtained from dosing an equimolar amount of tonabersat itself.

Example 1 was dosed according to this protocol at 6.43 mg/kg PO. Plasma levels of tonabersat were determined to be 53 ng/mL at 5 min and 576 ng/mL at 6 hrs showing conversion of the prodrug to tonabersat over this timecourse following oral dosing. This corresponds to an exposure of tonabersat following dosing of the prodrugs of 53% compared to the exposure obtained from dosing an equimolar amount of tonabersat itself.

Example 2 was dosed according to this protocol at 1.04 mg/kg IV. The plasma level of tonabersat was determined to be 2212 ng/mL at 5 min showing conversion of the prodrug to tonabersat following intravenous dosing. This corresponds to an exposure of tonabersat following dosing of the prodrugs of 45% compared to the exposure obtained from dosing an equimolar amount of tonabersat itself.

hERG Assay

Compounds of the invention were tested for inhibition of the human ether a go-go related gene (hERG) K⁺ channel using IonWorks patch clamp electrophysiology. 8 Point concentration-response curves were generated on two occasions using 3-fold serial dilutions from the maximum assay concentration (33 uM). Electrophysiological recordings were made from a Chinese Hamster Lung cell line stably expressing the full length hERG channel. Single cell ion currents were measured in the perforated patch clamp configuration (100 ug/mL amphoterocin) at room temperature using an IonWorks Quattro Instrument. The internal solution contained 140 mM KCl, 1 mM MgCl₂, 1 mM EGTA and 20 mM HEPES and was buffered to pH 7.3. The external solution contained 138 mM NaCl, 2.7 mM KCl, 0.9 mM CaCl₂, 0.5 mM MgCl₂, 8 mM Na₂HPO₄ and 1.5 mM KH₂PO₄, and was buffered to pH 7.3. Cells were clamped at a holding potential of 70 mV for 30 s and then stepped to +40 mV for 1 s. This was followed by a hyperpolarising step of 1 s to 30 mV to evoke the hERG tail current. This sequence was repeated 5 times at a frequency of 0.25 Hz. Currents were measured from the tall step at the 5^{th} pulse, and referenced to the holding current. Compounds were incubated for 6-7 min prior to a second measurement of the hERG signal using an identical pulse train. A minimum of 17 cells were required for each pIC50 curve fit. A control compound (quinidine) was used.

Example 1 was tested in line with the preceding experimental procedure and shown to have a hERG IC50 of >20 uM.

In an embodiment the compounds of the Invention have a hERG IC50 of >11 uM.

1. A compound according to formula (I) or a hydrate, solvate, or pharmaceutically acceptable salt thereof: [chemical structure]
wherein
Z₁, Z₂, and Z₃ are each independently selected from H, F, or Cl,
Q is O,
R² is H,
A is a direct bond, —C(O)O*—, C(R³)(R⁴)O*—, —C(O)NH* wherein the atom marked * is directly connected to R¹,
R³ and R⁴ are selected independently from H, fluoro, C₁₋₄ alkyl, or C₁₋₄ fluoroalkyl, or R³ and R⁴ together with the atom to which they are attached form a cyclopropyl group,
R¹ is selected from any one of the groups [1], [2], [3], [4], [5], [6], [7], [8], [9] or [10] wherein the atom marked ** is directly connected to A: [chemical structure]
n is 0, 1, 2, or 3,
R⁵ is hydrogen,
R⁶ is selected from —CH₂CH(OH)CH₂OH, or —CH₂CH₂R⁹,
R⁷ and R^{7b} are independently selected from H, C₁₋₄ alkyl, or C₁₋₄ fluoroalkyl,
R⁸ and R^{8b} are selected from:
(i) H, C₁₋₄ alkyl, or C₁₋₄ fluoroalkyl, or
(ii) the side chain of a natural or unnatural alpha-amino acid,
or R⁷ and R⁸ together with the atom to which they are attached form a C₃₋₇ carbocyclic ring,
R⁹ is selected from —N(R¹¹)(R¹²), or —N⁺ (R¹¹)(R¹²)(R¹³)X⁻, N(R¹¹)C(O)R¹⁴, —SO₃H, or —OP(O)(OH)₂,
wherein R¹¹, R¹², and R¹³ are independently selected from H, C₁₋₄ alkyl, or C₁₋₄ fluoroalkyl, or
R¹¹ and R¹² together with the nitrogen atom to which they are attached form a 4-7 membered heterocyclic ring optionally substituted with one or more groups selected from H, fluoro, C₁₋₄ alkyl, C₁₋₄ fluoroalkyl, C₁₋₄ alkoxy, or —C(O)R₃,
or in the case where R¹ is group [7], R⁹ is —NR¹¹R¹², wherein R¹¹ is hydrogen, C₁₋₄ alkyl, or C₁₋₄ fluoroalkyl, and R¹² is C₁₋₄ alkyl, or C₁₋₄ fluoroalkyl, and R¹² joins together with R^{8b} such that R¹² and R^{8b} together with the nitrogen to which R¹² is attached form a 5 or 6 membered cyclic amine group,
R¹⁴ is H, C₁₋₄ alkyl, or C₁₋₄ fluoroalkyl,
X⁻ is a pharmaceutically acceptable anion,
R¹⁵ is 3-pyridyl or 1,4-dihydro-1-methyl-pyridin-3-yl,
Y is —O—, —CH₂—, —N(H)—, or —N(CH₃)—.

2. The compound of claim 1 wherein Z₁ is C₁, Z₂ is F or hydrogen, and Z₃ is hydrogen.

3. The compound of claim 1 wherein R³ and R⁴ are hydrogen

4. The compound of claim 1 wherein R¹¹, R¹², and R¹³ are independently methyl or ethyl.

5. The compound of claim 1 wherein R¹¹ and R¹² together with the nitrogen atom to which they are attached form a 5 or 6 membered cyclic amino group.

6. The compound of claim 5 wherein the cyclic amino group is selected from morpholine, pyrrolidine, piperidine, or piperazine.

7. The compound of claim 5 wherein the cyclic amino group is substituted with one or more substituents selected from chloro, fluoro, methyl, isopropyl, —OCH₃, or —C(O)CH₃.

8. The compound of claim 1 wherein R⁷ is hydrogen and R⁸ is the side chain of a natural or unnatural amino acid.

9. The compound of claim 1 wherein R^{7b} is hydrogen and R^{8b} is the side chain of a natural or unnatural amino acid.

10. The compound of claim 1 wherein the side chain of the natural or unnatural amino acid is selected from —CH(CH₃)₂, —(CH₂)₃CH₂NH₂, —CH(CH₃)(CH₂CH₂CH₃), or —CH₂CH(CH₃)₂.

11. The compound of claim 1 wherein R⁷ and R⁸ are both hydrogen.

12. The compound of claim 1 wherein R^{7b} and R^{8b} are both hydrogen.

13. The compound of claim 1 wherein R⁶ is selected from —CH₂CH(OH)CH₂OH, —CH₂CH₂NR¹¹R¹², or —CH₂CH₂NR¹¹R¹²R¹³X⁻.

14. The compound of claim 1 wherein R¹ is selected from [4A], [4B], [4C], [4D], [5A], or [5B]:

[chemical structure]

15-24. (canceled)

25. A pharmaceutical composition comprising a compound of claim 1, together with one or more pharmaceutically acceptable carriers and/or excipients.

26. The pharmaceutical composition of claim 25 formulated as a liquid for intravenous dosage.

27. The pharmaceutical composition of claim 25 formulated as a solid for oral dosage.

28. A method of treatment of a disease or medical condition which benefits from inhibition of gap junction activity, comprising administering to a subject suffering from such disease or condition and effective amount of a compound as claimed in claim 1.

29. The method of treatment of claim 28 wherein the disease or condition is selected from among migraine, aura with or without migraine, epilepsy, non-epileptic seizures, cerebrovascular accidents, spinal cord vascular accidents, pain, neurodegenerative disease, and cardiovascular disease.

30. The method of treatment of claim 29 wherein the disease or condition is a cerebrovascular accident including stroke, intracranial haemorrhage (including or traumatic brain injury, epidural hematoma, subdural hematoma and subarachnoid haemorrhage), and intra-cerebral haemorrhage.

31. The method of treatment of claim 29 wherein the disease or condition is a spinal cord vascular accident arising from trauma, epidural hematoma, subdural hematoma or subarachnoid haemorrhage.

32. The method of treatment of claim 29 wherein the disease or condition is hyperalgesia caused by damage to sensory neurons, including diabetic neuropathy, polyneuropathy, cancer pain, fibromyalgia, myofascial pain, post herpetic neuralgia, spinal stenosis, HIV pain, post-operative pain, and post-trauma pain.

33. The method of treatment of claim 29 wherein the disease or condition is inflammatory pain, including pain associated with osteoarthritis, rheumatoid arthritis, sciatica/radiculopathy, pancreatitis, and tendonitis.

34. The method of treatment of claim 29 wherein the disease or condition is a neurodegenerative disease, including Alzheimer's Disease, Parkinson's Disease, Huntington's Disease and Amyotrophic Lateral Sclerosis.
